(19) United States
(12) Patent Application Publication
(10) Pub. No.: US 2014/0378995 A1
(43) Pub. Date: Dec. 25, 2014

(54) METHOD AND SYSTEM FOR ANALYZING A TASK TRAJECTORY

(71) Applicants: Intuitive Surgical Operations, Inc., Sunnyvale, CA (US); The Johns Hopkins University, Baltimore, MD (US)

(72) Inventors: Rajesh Kumar, Baltimore, MD (US); Gregory D. Hager, Baltimore, MD (US); Amod S. Jog, Baltimore, MD (US); Yixin Gao, Baltimore, MD (US); May Liu, Sunnyvale, CA (US); Simon Peter DiMaio, Sunnyvale, CA (US); Brandon Itkowitz, Sunnyvale, CA (US); Myriam Curet, Los Altos Hills, CA (US)

(73) Assignees: Intuitive Surgical Operations, Inc., Sunnyvale, CA (US); The Johns Hopkins University, Baltimore, MD (US)

(21) Appl. No.: 14/115,092

(22) PCT Filed: May 7, 2012
(86) PCT No.: PCT/US12/36822

§ 371 Date: Jul. 29, 2014

Related U.S. Application Data
(60) Provisional application No. 61/482,831, filed on May 5, 2011.

(51) Int. Cl. A61B 19/00; A61B 5/06

(52) U.S. Cl. CPC A61B 19/2203; A61B 5/065
USPC 606/130

(57) ABSTRACT

A computer-implemented method of analyzing a sample task trajectory including obtaining, with one or more computers, position information of an instrument in the sample task trajectory, obtaining, with the one or more computers, pose information of the instrument in the sample task trajectory, comparing, with the one or more computers, the position information and the pose information for the sample task trajectory with reference position information and reference pose information of the instrument for a reference task trajectory, determining, with the one or more computers, a skill assessment for the sample task trajectory based on the comparison, and outputting, with the one or more computers, the determined skill assessment for the sample task trajectory.

### CROSS-REFERENCE OF RELATED APPLICATION

This application claims priority to U.S. Provisional Application No. 61/482,831 filed May 5, 2011, the entire contents of which are hereby incorporated by reference.

This invention was made with Government support of Grant No. 1R21EB009143-01A1, awarded by the National Institute of Health and Grant Nos. 0941362 and 0931805, awarded by the National Science Foundation. The U.S. Government has certain rights in this invention.

### BACKGROUND

1. Field of Invention

The current invention relates to analyzing a trajectory, and more particularly to analyzing a task trajectory.

2. Discussion of Related Art

The contents of all references, including articles, published patent applications and patents referred to anywhere in this specification are hereby incorporated by reference.

With the widespread use of the nearly two thousand da Vinci surgical systems [Badani, K K and Kaul, S. and Menon, M. Evolution of robotic radical prostatectomy: assessment after 2766 procedures. Cancer, 110(9):1951-1958, 2007] for robotic surgery in urology [Boggess, J. F. Robotic surgery in gynecologic oncology: evolution of a new surgical paradigm. Journal of Robotic Surgery, 1(1):31-37, 2007; Chang, L. and Satava, R M and Pellegrini, C A and Sinanan, M N. Robotic surgery: identifying the learning curve through objective measurement of skill. Surgical endoscopy, 17(11):1744-1748, 2003], gynaecology [Chitwood Jr, W. R. Current status of endoscopic and robotic mitral valve surgery. The Annals of thoracic surgery, 79(6):2248-2253, 2005], cardiac surgery [Cohen, Jacob. A Coefficient of Agreement for Nominal Scales. Educational and Psychological Measurement, 20(1):37-46, 1960; Simon DiMaio and Chris Hasser. The da Vinci Research Interface. 2008 MICCAI Workshop—Systems and Architectures for Computer Assisted Interventions, Midas Journal, http://hdl.handle.net/1926/1464, 2008] and other specialties, an acute need for training, including simulation based training has arisen. A da Vinci telesurgical system includes a console containing an auto-stereoscopic viewer, system configuration panels, and master manipulators which control a set of disposable wristed surgical instruments mounted on a separate set of patient side manipulators. A surgeon teleoperates these instruments while viewing the stereo output of an endoscopic camera mounted on one of the instrument manipulators. The da Vinci surgical system is a complex man-machine interaction system. As with any complex system, it requires a considerable amount of practice and training to achieve proficiency.

Prior studies have shown that training in robotic surgery allows laparoscopic surgeons to perform robotic surgery tasks more efficiently compared to standard laparoscopy [Duda, Richard O. and Hart, Peter E. and Stork, David G. Pattern Classification (2nd Edition). Wiley-Interscience, 2000], and that skill acquisition in robotic surgery is dependent on practice and evaluation [Grantcharov, T P and Kristiansen, V B and Bendix, J. and Bardram, L. and Rosenberg, J. and Funch-Jensen, P. Randomized clinical trial of virtual reality simulation for laparoscopic skills training British Journal of Surgery, 91(2):146-150, 2004]. Literature also frequently notes the need for standardized training and assessment methods for minimally invasive surgery [Hall, M and Frank, E and Holmes, G and Pfahringer, B and Reutemann, P and Witten, I. H. The WEKA Data Mining Software: An Update. SIGKDD Explorations, 11, 2009; Jog, A and Itkowitz, B and Liu, M and DiMaio, S and Hager, G and Curet, M and Kumar, R. Towards integrating task information in skills assessment for dexterous tasks in surgery and simulation. IEEE International Conference on Robotics and Automation, pages 5273-5278, 2011]. Studies on training with real models [Judkins, T. N. and Oleynikov, D. and Stergiou, N. Objective evaluation of expert and novice performance during robotic surgical training tasks. Surgical Endoscopy, 23(3):590-597, 2009] have also shown that robotic surgery though complex, is equally challenging when presented as a new technology to novice and expert laparoscopic surgeons.

Simulation and virtual reality training [Kaul, S. and Shah, N. L. and Menon, M. Learning curve using robotic surgery. Current Urology Reports, 7(2):125-129, 2006] have long been used in robotic surgery. Simulation-based training and testing programs are already being used for assessing operational technical skill, and non-technical skills in some specialties [Kaul, S. and Shah, N. L. and Menon, M. Learning curve using robotic surgery. Current Urology Reports, 7(2):125-129, 2006; Kenney, P. A. and Wszolek, M. F. and Gould, J. J. and Libertino, J. A. and Moinzadeh, A. Face, content, and construct validity of dV-trainer, a novel virtual reality simulator for robotic surgery. Urology, 73(6):1288-1292, 2009]. Virtual reality trainers with full procedure tasks have been used to simulate realistic procedure level training and measure the effect of training by observing performance in the real world task [Kaul, S. and Shah, N. L. and Menon, M. Learning curve using robotic surgery. Current Urology Reports, 7(2):125-129, 2006; Kumar, R and Jog, A and Malpani, A and Vagvolgyi, B and Yuh, D and Nguyen, H and Hager, G and Chen, C C G. System operation skills in robotic surgery trainees. The International Journal of Medical Robotics and Computer Assisted Surgery, accepted, 2011; Lendvay, T. S. and Casale, P. and Sweet, R. and Peters, C. Initial validation of a virtual-reality robotic simulator. Journal of Robotic Surgery, 2(3):145-149, 2008; Lerner, M. A. and Ayalew, M. and Peine, W. J. and Sundaram, C. P. Does Training on a Virtual Reality Robotic Simulator Improve Performance on the da Vinci Surgical System?. Journal of Endourology, 24(3):467, 2010]. Training using simulated tasks can be easily replicated and repeated. Simulation based robotic training is also a more cost effective way of training as it does not require real instruments or training pods. Bench top standalone robotic surgery trainers are currently in advanced evaluation [Lin, H. C. and Shafran, I. and Yuh, D. and Hager, G. D. Towards automatic skill evaluation: Detection and segmentation of robot-assisted surgical motions. Computer Aided Surgery, 11(5):220-230, 2006; Moorthy, K. and Munz, Y. and Dosis, A. and Hernandez, J. and Martin, S. and Bello, F. and Rockall, T. and Darzi, A. Dexterity enhancement with robotic surgery. Surgical Endoscopy, 18:790-795, 2004. 10.1007/s00464-003-8922-2]. Intuitive Surgical Inc. has also developed the da Vinci Skills Simulator to allow training on simulated tasks in an immersive virtual environment.

FIG. 1 illustrates a simulator for simulating a task along with a display of a simulation and a corresponding performance report according to an embodiment of the current invention. The simulator use a surgeon's console from the da Vinci system integrated with a software suite to simulate the instrument and the training environment. The training exercises can be configured for many levels of difficulty. Upon completion of a task, the user receives a report describing performance metrics and a composite score is calculated from these metrics.

As all hand and instrument motion can be captured in both real and simulation based robotic training, corresponding basic task statistics such as time to complete a task, instrument and hand distances traveled, and volumes of hand or instrument motion have been used as common performance metrics [Lin, H. C. and Shafran, I. and Yuh, D. and Hager, G. D. Towards automatic skill evaluation: Detection and segmentation of robot-assisted surgical motions. Computer Aided Surgery, 11(5):220-230, 2006]. This motion data may correspond to a trajectory of an instrument while completing the task. This motion data can be accessed through an application programming interface (API) [Munz, Y. and Kumar, B. D. and Moorthy, K. and Bann, S. and Darzi, A. Laparoscopic virtual reality and box trainers: is one superior to the other?. Surgical Endoscopy, 18:485-494, 2004. 10.1007/s00464-003-9043-7]. The API is an Ethernet interface that streams the motion variables including joint, Cartesian and torque data of all manipulators in the system in real-time. The data streaming rate is configurable and can be as high as 100 Hz. The da Vinci system also provides for acquisition of stereo endoscopic video data from spare outputs.

Prior evaluation studies have primarily focused on face, content, and construct validity of these simple statistics [Quinlan, J. Ross. C4.5: Programs for Machine Learning. Morgan Kaufmann Publishers Inc., San Francisco, Calif., USA, 1993; Reiley, Carol and Lin, Henry and Yuh, David and Hager, Gregory. Review of methods for objective surgical skill evaluation. Surgical Endoscopy, :1-11, 2010. 10.1007/s00464-010-1190-z] reported by the evaluation system of the simulator based on such motion data. Although these statistics may be coarsely related to the task performance, they do not provide any insight into individual task performance, or any method for effective comparison between two task performances. They are also not useful for providing specific or detailed user feedback. Note, for example, that the task completion time is not a good training metric. It is the task outcome or quality that should be the training focus.

There is thus a need for improved analysis of a task trajectory.

### SUMMARY

A computer-implemented method of analyzing a sample task trajectory including obtaining, with one or more computers, position information of an instrument in the sample task trajectory, obtaining, with the one or more computers, pose information of the instrument in the sample task trajectory, comparing, with the one or more computers, the position information and the pose information for the sample task trajectory with reference position information and reference pose information of the instrument for a reference task trajectory, determining, with the one or more computers, a skill assessment for the sample task trajectory based on the comparison, and outputting, with the one or more computers, the determined skill assessment for the sample task trajectory.

A system for analyzing a sample task trajectory including a controller configured to receive motion input from a user for an instrument for the sample task trajectory and a display configured to output a view based on the received motion input. The system further includes a processor configured to obtain position information of the instrument in the sample task trajectory based on the received motion input, obtain pose information of the instrument in the sample task trajectory based on the received motion input, compare the position information and the pose information for the sample task trajectory with reference position information and reference pose information of the instrument for a reference task trajectory, determine a skill assessment for the sample task trajectory based on the comparison, and output the skill assessment.

One or more tangible non-transitory computer-readable storage media for storing computer-executable instructions executable by processing logic, the media storing one or more instructions. The one or more instructions are for obtaining position information of an instrument in the sample task trajectory, obtaining pose information of the instrument in the sample task trajectory, comparing the position information and the pose information for the sample task trajectory with reference position information and reference pose information of the instrument for a reference task trajectory, determining a skill assessment for the sample task trajectory based on the comparison, and outputting the skill assessment for the sample task trajectory.

### BRIEF DESCRIPTION OF THE DRAWINGS

Further objectives and advantages will become apparent from a consideration of the description, drawings, and examples.

FIG. 1 illustrates a simulator for simulating a task along with a display of a simulation and a corresponding performance report according to an embodiment of the current invention.

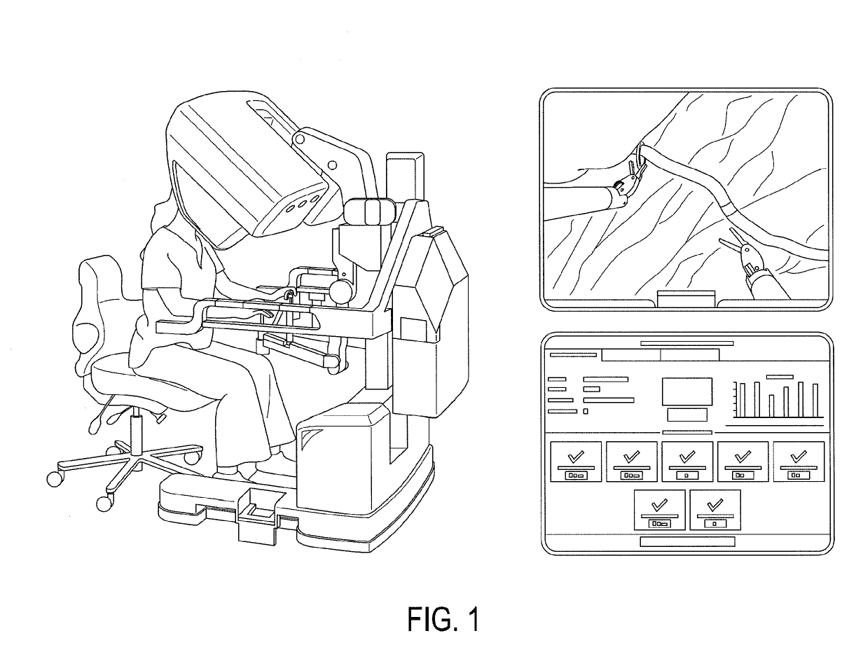

FIG. 2 illustrates a block diagram of a system according to an embodiment of the current invention.

FIG. 3 illustrates an exemplary process flowchart for analyzing a sample task trajectory according to an embodiment of the current invention.

FIG. 4 illustrates a surface area defined by an instrument according to an embodiment of the current invention.

FIGS. 5A and 5B illustrate a task trajectory of an expert and a task trajectory of a novice, respectively, according to an embodiment of the current invention.

FIG. 6 illustrates a pegboard task according to an embodiment of the current invention.

FIG. 7 illustrates a ring walk task according to an embodiment of the current invention.

FIG. 8 illustrates task trajectories during the ring walk task according to an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

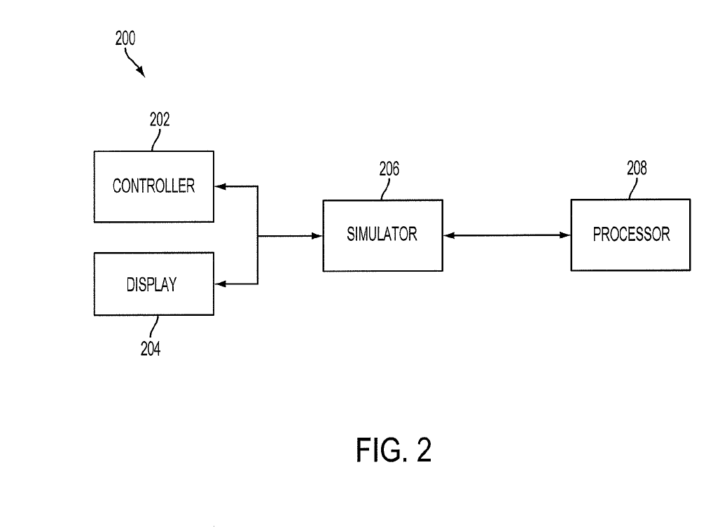

FIG. 2 illustrates a block diagram of system 200 according to an embodiment of the current invention. System 200 includes controller 202, display 204, simulator 206, and processor 208.

Controller 202 may be a configured to receive motion input from a user. Motion input may include input regarding motion. Motion may include motion in three dimensions of an instrument. An instrument may include a tool used for a task. The tool may include a surgical instrument and the task may include a surgical task. For example, controller 202 may be a master manipulator of a da Vinci telesurgical system whereby a user may provide input for an instrument manipulator of the system which includes a surgical instrument. The motion input may be for a sample task trajectory. The sample task trajectory may be a trajectory of an instrument during a task based on the motion input where the trajectory is a sample which is to be analyzed.

Display 204 may be configured to output a view based on the received motion input. For example, display 204 may be a liquid crystal display (LCD) device. A view which is output on display 204 may be based on a simulation of a task using the received motion input.

Simulator 206 may be configured to receive the motion input from controller 202 to simulate a sample task trajectory based on the motion input. Simulator 206 may be configured to further generate a view based on the receive motion input. For example, simulator 206 may generate a view of an instrument during a surgical task based on the received motion input. Simulator 206 may provide the view to display 204 to output the view.

Processor 208 may be a processing unit adapted to obtain position information of the instrument in the sample task trajectory based on the received motion input. The processing unit may be a computing device, e.g., a computer. Position information may be information on the position of the instrument in a three dimensional coordinate system. Position information may further include a timestamp identifying the time at which the instrument is at the position. Processor 208 may receive the motion input and calculate position information or processor 208 may receive position information from simulator 206.

Processor 208 may be further adapted to obtain pose information of the instrument in the sample task trajectory based on the received motion input. Pose information may include information on the orientation of the instrument in a three dimensional coordinate system. Pose information may correspond to roll, pitch, and yaw information of the instrument. The roll, pitch, and yaw information may correspond to a line along a last degree of freedom of the instrument. The pose information may be represented using at least one of a position vector and a rotation matrix in a conventional homogeneous transformation framework, three angles of pose and three elements of a position vector in a standard axis-angle representation, or a screw axis representation. Pose information may further include a timestamp identifying the time at which the instrument is at the pose. Processor 208 may receive the motion input and calculate pose information or processor 208 may receive pose information from simulator 206.

Processor 208 may be further configured to compare the position information and the pose information for the sample task trajectory with reference position information and reference pose information of the instrument for a reference task trajectory. The reference task trajectory may be a trajectory of an instrument during a task where the trajectory is a reference to be compared to a sample trajectory. For example, reference task trajectory could be a trajectory made by an expert. Processor 208 may be configured to determine a skill assessment for the sample task trajectory based on the comparison and output the skill assessment. A skill assessment may be a score and/or a classification. A classification may be a binary classification between novice and expert.

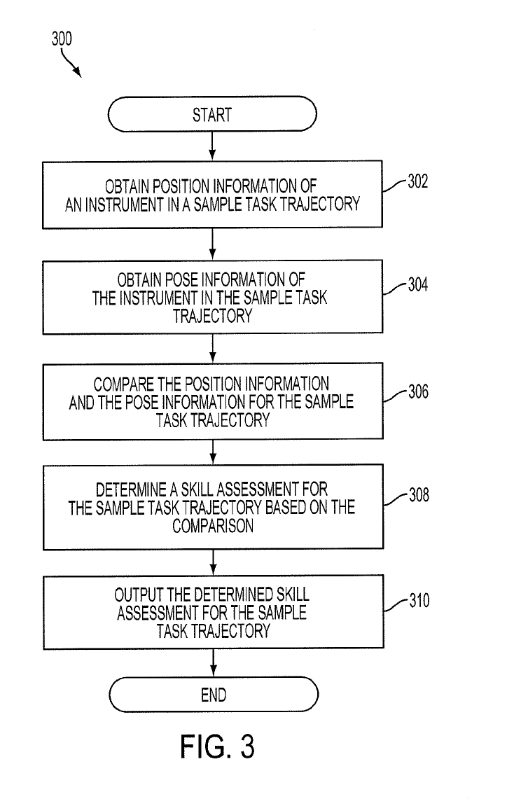

FIG. 3 illustrates exemplary process flowchart 300 for analyzing a sample task trajectory according to an embodiment of the current invention. Initially, processor 208 may obtain position information of an instrument in a sample task trajectory (block 302) and obtain pose information of the instrument in the sample task trajectory (block 304). As discussed, processor 208 may receive the motion input and calculate position and pose information or processor 208 may receive position and pose information from simulator 206.

In obtaining the position information and pose information, processor 208 may also filter the position information and pose information. For example, processor 208 may exclude information corresponding to non-important motion. Processor 208 may detect the importance or task relevance of position and pose information based on detecting a portion of the sample task trajectory which was outside a field of view of the user or identifying a portion of the sample task trajectory which is unrelated to a task. For example, processor 208 may exclude movement made to bring an instrument into the field of view shown on display 204 as this movement may be unimportant to the quality of the task performance. Processor 208 may also consider information corresponding to when an instrument is touching tissue as relevant.

Processor 208 may compare the position information and the pose information for the sample task trajectory with reference position information and reference pose information (block 306).

The position information and the pose information of the instrument for the sample task trajectory may be based on the corresponding orientation and location of a camera. For example, the position information and the pose information may be in a coordinate system referenced to the orientation and location of a camera of a robot including the instrument. In comparing, processor 208 may transform the position information of the instrument and the pose information of the instrument from a coordinate system based on the camera to a coordinate system based on the reference task trajectory. For example, processor 208 may correspond position information of the instrument in a sample task trajectory with reference position information for a reference task trajectory and identify the difference between the pose information of the instrument and reference pose information based on the correspondence.

The correspondence between the trajectory points may also be established by using methods such as dynamic time warping.

Processor 208 may alternatively transform the position information of the instrument and the pose information of the instrument from a coordinate system based on the camera to a coordinate system based on a world space. The world space may be based on setting a fixed position as a zero point and setting coordinates in reference to the fixed position. The reference position information of the instrument and the reference pose information of the instrument may also be transformed to a coordinate system based on a world space. Processor 208 may compare the position information of the instrument and the pose information of the instrument in the coordinate system based on the world space with the reference position information of the instrument and the reference pose information in the coordinate system based on the world space. In another example, processor 208 may transform the information to a coordinate system based on a dynamic point. For example, the coordinate system may be based on a point on a patient where the point moves as the patient moves.

In comparing, processor 208 may also correspond the sample task trajectory and reference task trajectory based on progress in the task. For example, processor 208 may identify the time at which 50% of the task is completed during the sample task trajectory and the time at which 50% of the task is completed during the reference task trajectory. Corresponding based on progress may account for differences in the trajectories during the task. For example, processor 208 may determine that the sample task trajectory is performed at 50% of the speed that the reference task trajectory is performed. Accordingly, processor 208 may compare the position and pose information corresponding to 50% task completion during the sample task trajectory with the reference position and pose information corresponding to 50% task completion during the reference task trajectory.

In comparing, processor 208 may further perform comparison based on surface area spanned by a line along an instrument axis of the instrument during the sample task trajectory. Processor 208 may compare the calculated surface area with a corresponding surface area spanned during the reference task trajectory. Processor 208 may calculate the surface area based on generating a sum of areas of consecutive quadrilaterals defined by the line sampled at one or more of time intervals, equal instrument tip distances, or equal angular or pose separation.

Processor 208 may determine a skill assessment for the sample task trajectory based on the comparison (block 308). In determining the skill assessment, processor 208 may classify the sample task trajectory into a binary skill classification for users of a surgical robot based on the comparison. For example, processor 208 may determine that a sample task trajectory corresponds to either an unproficient user or a proficient user. Alternatively, processor 208 may determine the skill assessment is a score of 90%.

In determining the skill assessment, processor 208 may calculate and weigh metrics based on one or more of the total surface spanned by a line along an instrument axis, total time, excessive force used, instrument collisions, total out of view instrument motion, range of the motion input, and critical errors made. These metrics may be equally weighted or unequally weighted. Adaptive thresholds may also be determined for classifying. For example, processor 208 may be provided task trajectories that are identified as those corresponding to proficient users and task trajectories that are identified as those corresponding to non-proficient users. Processor 208 may then adaptively determine thresholds and weights for the metrics which correctly classify the trajectories based on the known identifications of the trajectories.

Process flowchart 300 may also analyze a sample task trajectory based on velocity information and gripper angle information. Processor 208 may obtain velocity information of the instrument in the sample task trajectory and obtain gripper angle information of the instrument in the sample trajectory. When processor 208 compares the position information and the pose information, processor 208 may further compare the velocity information and gripper angle information with reference velocity information and reference gripper angle information of the instrument for the reference task trajectory.

Processor 208 may output the determined skill assessment for the sample task trajectory (block 310). Processor 208 may output the determined skill assessment via an output device. An output device may include at least one of display 104, a printer, speakers, etc.

Tasks may also involve the use of multiple instruments which may be separately controlled by a user. Accordingly, a task may include multiple trajectories where each trajectory corresponds to an instrument used in the task. Processor 208 may obtain position information and pose information for multiple sample trajectories during a task, obtain reference position information and reference pose information for multiple reference trajectories during a task to compare and determine a skill assessment for the task.

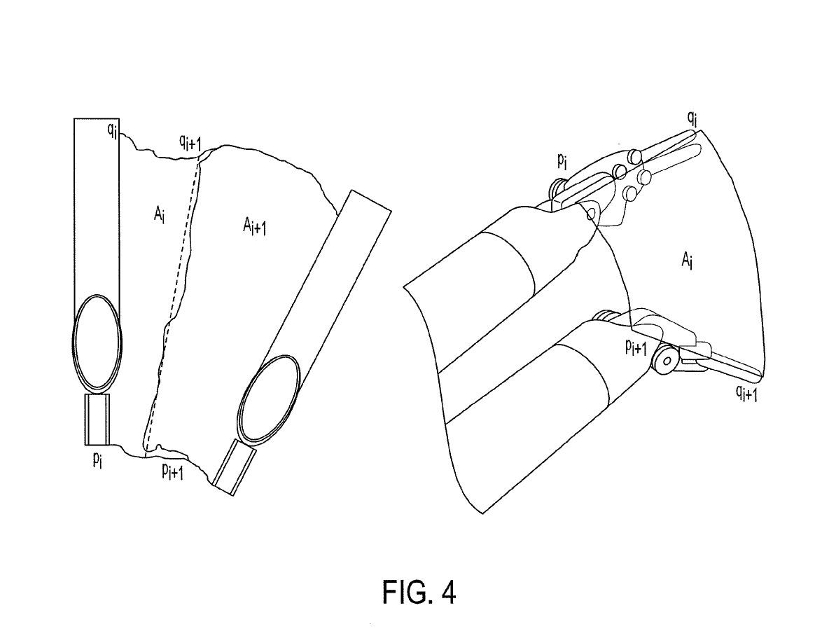

FIG. 4 illustrates a surface area defined by an instrument according to an embodiment of the current invention. As illustrated, a line may be defined by points p, and q, along an axis of the instrument. Point qi may correspond with the kinematic tip of the instrument and q, may correspond to a point on the gripper of the instrument. A surface area may be defined based on the area covered by the line between a first sample time during a sample task trajectory and a second sample time during the sample task trajectory. As shown in FIG. 4, surface area A, is a quadrilateral defined by points qᵢ, pᵢ, qᵢ₊₁, and pᵢ₊₁.

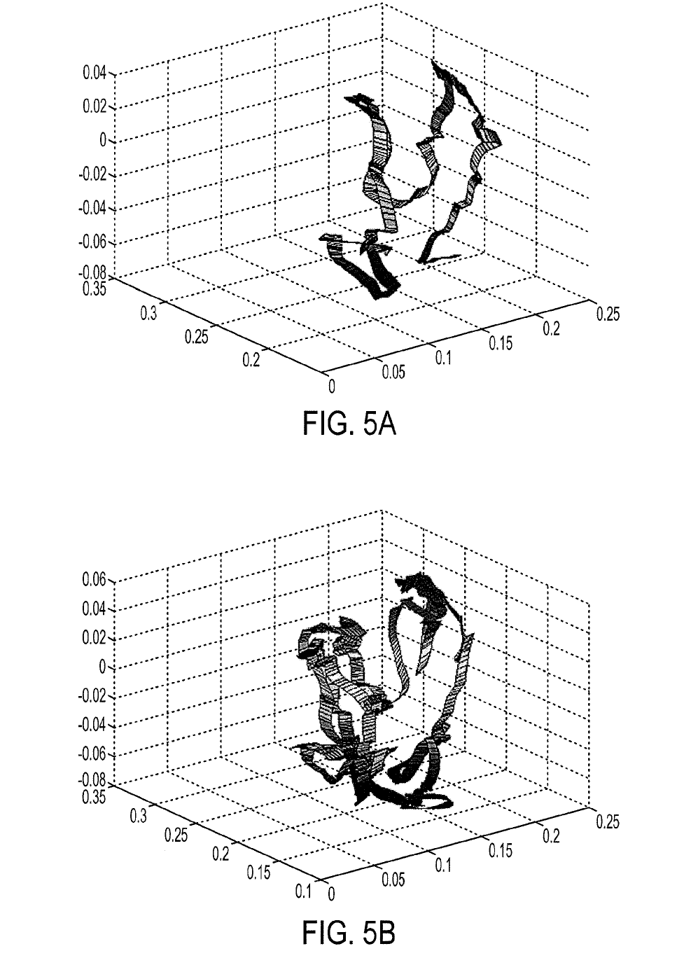

FIGS. 5A and 5B illustrate a task trajectory of an expert and a task trajectory of a novice, respectively, according to an embodiment of the current invention. The task trajectories shown may correspond to the surface area spanned by a line along an instrument axis of the instrument during the task trajectory. Both trajectories have been transformed to a shared reference frame (for example the robot base frame or the “world” frame) so they can be compared, and correspondences established. The surface area (or “ribbon”) spanned by the instrument can be configurable depending upon task, task time, or user preference aimed at distinguishing users of varying skill.

### Example

### I. Introduction

Published studies have explored skill assessment using the kinematic data from the da Vinci API [Judkins, T. N. and Oleynikov, D. and Stergiou, N. Objective evaluation of expert and novice performance during robotic surgical training tasks. Surgical Endoscopy, 23(3):590-597, 2009; Lin, H. C. and Shafran, I. and Yuh, D. and Hager, G. D. Towards automatic skill evaluation: Detection and segmentation of robot-assisted surgical motions. Computer Aided Surgery, 11(5):220-230, 2006; Sarle, R. and Tewari, A. and Shrivastava, A. and Peabody, J. and Menon, M. Surgical robotics and laparoscopic training drills. Journal of Endourology, 18(1):63-67, 2004] for training tasks performed on training pods. Judkins et al [Judkins, T. N. and Oleynikov, D. and Stergiou, N. Objective evaluation of expert and novice performance during robotic surgical training tasks. Surgical Endoscopy, 23(3):590-597, 2009] used task completion time, distance traveled, speed, and curvature for ten subjects to distinguish experts from novices in simple tasks. The novices performed as well as the experts after a small number of trials. Lin et al [Lin, H. C. and Shafran, I. and Yuh, D. and Hager, G. D. Towards automatic skill evaluation: Detection and segmentation of robot-assisted surgical motions. Computer Aided Surgery, 11(5):220-230, 2006] used 72 kinematic variables skill classification for a four throw suturing task, which was decomposed into labeled sequence of surgical labels. Other analysis has used data driven models like Hidden Markov models (HMM) and motion data with labeled surgical gestures to assess surgical skill [Reiley, Carol and Lin, Henry and Yuh, David and Hager, Gregory. Review of methods for objective surgical skill evaluation. Surgical Endoscopy, :1-11, 2010. 10.1007/s00464-010-1190-z; Varadarajan, Balakrishnan and Reiley, Carol and Lin, Henry and Khudanpur, Sanjeev and Hager, Gregory. Data-Derived Models for Segmentation with Application to Surgical Assessment and Training In Yang, Guang-Zhong and Hawkes, David and Rueckert, Daniel and Noble, Alison and Taylor, Chris, editors, Medical Image Computing and Computer-Assisted Intervention \hat{d} [symbol] “MICCAI 2009 in Lecture Notes in Computer Science, pages 426-434. Springer Berlin/Heidelberg, 2009].

Robotic surgery motion data has been analyzed for skill classification, establishment of learning curves, and training curricula development [Jog, A and Itkowitz, B and Liu, M and DiMaio, S and Hager, G and Curet, M and Kumar, R. Towards integrating task information in skills assessment for dexterous tasks in surgery and simulation. IEEE International Conference on Robotics and Automation, pages 5273-5278, 2011; Kumar, R and Jog, A and Malpani, A and Vagvolgyi, B and Yuh, D and Nguyen, H and Hager, G and Chen, C C G. System operation skills in robotic surgery trainees. The International Journal of Medical Robotics and Computer Assisted Surgery, :accepted, 2011; Yuh, D D and Jog, A and Kumar, R. Automated Skill Assessment for Robotic Surgical Training 47th Annual Meeting of the Society of Thoracic Surgeons, San Diego, Calif., pages poster, 2011].

Variability in task environment and execution by different subjects, and a lack of environment models or task quality assessment for real task pod based training has meant previous analysis has focused on establishing lower variability in expert task executions, and classification of users based on their trajectories in the Euclidean space. These limitations is being addressed to some extent by acquiring structured assessment by multiple experts [Yuh, D D and Jog, A and Kumar, R. Automated Skill Assessment for Robotic Surgical Training 47th Annual Meeting of the Society of Thoracic Surgeons, San Diego, Calif., pages poster, 2011], and by structuring the environment with fiducials to automatically capture instrument/environment interactions.

By contrast, the simulated environment provides complete information about both the task environment state, as well as the task/environment interactions. Simulated environments are tailor made to compare the performance of multiple users because of the reproducibility. Since tasks can be readily repeated, a trainee is more likely to perform a large number of unsupervised trials, and metrics of performance are needed to identify if acceptable proficiency has been achieved or if more repetitions of a particular training task would be helpful. The metrics reported above measure progress, but do not contain sufficient information to assess proficiency.

In this example skill proficiency classification for simulated robotic surgery training tasks is attempted. Given motion data from the simulated environment, a new metric for describing the performance in a particular trial is described along with alternate workspaces for skill classification methods. Finally, statistical classification methods are applied in this alternate workspace to show promising proficiency classification for both simple, and complex robotic surgery training tasks.

### II. Methods

The MIMIC dV-Trainer [Kenney, P. A. and Wszolek, M. F. and Gould, J. J. and Libertino, J. A. and Moinzadeh, A. Face, content, and construct validity of dV-trainer, a novel virtual reality simulator for robotic surgery. Urology, 73(6):1288-1292, 2009; Lendvay, T. S. and Casale, P. and Sweet, R. and Peters, C. Initial validation of a virtual-reality robotic simulator. Journal of Robotic Surgery, 2(3):145-149, 2008; Lerner, M. A. and Ayalew, M. and Peine, W. J. and Sundaram, C. P. Does Training on a Virtual Reality Robotic Simulator Improve Performance on the da Vinci Surgical System?. Journal of Endourology, 24(3):467, 2010] robotic surgical simulator (MIMIC Technologies, Inc., Seattle, Wash.) provides a virtual task trainer for the da Vinci surgical system with a low cost table-top console. While this console is suitable for bench-top training, it lacks the man-machine interface of the real da Vinci console. The da Vinci Skills Simulator removes these limitations by integrating the simulated task environment with the master console of a da Vinci Si system. The virtual instruments are manipulated using the master manipulators as in the real system.

The simulation environment provides motion data similar to the API stream [Simon DiMaio and Chris Hasser. The da Vinci Research Interface. 2008 MICCAI Workshop—Systems and Architectures for Computer Assisted Interventions, Midas Journal, http://hdl.handle.net/1926/1464, 2008] provided by the da Vinci surgical system. The motion data describes the motion of the virtual instruments, master handles and the camera. Streamed motion parameters include the Cartesian pose, linear and angular velocities, gripper angles and joint positions. The API may be sampled at 20 Hz for experiments and the timestamp (1 dimension), instrument Cartesian position (3 dimensions), orientation (3 dimensions), velocity (3 dimensions), and gripper position (1 dimension) extracted in a 10 dimensional vector for each of the instrument manipulators and the endoscopic camera manipulator.

The instrument pose is provided in the camera coordinate frame, which can be transformed into a static “world” frame by a rigid transformation with the endoscopic camera frame. Since this reference frame is shared across all the trials and for the virtual environment models being manipulated, trajectories may be anazlyed across the systems reconfiguration and trials.

For a given trajectory, let pₜ and pₜ₊₁ be two consecutive 3D points. The line distance p_{D} traveled may be calculated as:

$${p}_{D} = {\sum }_{i}^{ } d ( {p}_{t} , {p}_{t + 1} ) \qquad ( 1 )$$

where d( . . . ) is the Euclidean distance between two points. The corresponding task completion time p_{T} can also be directly measured from the timestamps. The simulator reports these measures at the end of a trial, including the line distance accumulated over the trajectory as a measure of motion efficiency [Lendvay, T. S. and Casale, P. and Sweet, R. and Peters, C. Initial validation of a virtual-reality robotic simulator. Journal of Robotic Surgery, 2(3):145-149, 2008].

The line distance may only use the instrument tip position, and not the full 6 DOF pose. In any dexterous motion that involves reorientation (most common instrument motions) using just the tip trajectory is not sufficient to capture the differences in skill. To capture the pose, the surface generated by a “brush” consisting of the tool clevis point at time t, pₜ and another point qₜ at a distance of 1 mm from the clevis along the instrument axis is traced. If the area of the quadrilateral generated by pₜ, qₜ, and and qₜ₊₁ is 4, then the surface area R_{A} for the entire trajectory can be computed as:

$${R}_{A} = {\sum }_{t}^{ } {A}_{t} \qquad ( 2 )$$

This measure may be called a “ribbon” area measure, and it is indicative of efficient pose management during the training task. Skill classification using adaptive threshold on simple statistical measures above also gives us baseline proficiency classification performance.

An adaptive threshold may be computed using the C4.5 algorithm [Quinlan, J. Ross. C4.5: Programs for Machine Learning. Morgan Kaufmann Publishers Inc., San Francisco, Calif., USA, 1993] by creating a single root decision tree node with two child nodes. For n metric values (x) corresponding to n trials and a given proficiency label for each of the trial, the decision tree classifier operates on the one dimensional data x₁, x₂, . . . , xₙ and an associated binary attribute label data m₁, m₂, . . . , mₙ (here, 0—trainee or 1—proficient). The input data is split based on a threshold xₜₕ on this attribute that maximizes the normalized information gain. The left node then contains all the samples with xᵢ≦xₜₕ and the right node with all samples xᵢ>xₜₕ.

Statistical Classification: For statistical proficiency classification, the instrument trajectory (L) for left and right instruments (10 dimensions each) may be sampled at regular distance intervals. The resulting 20 dimensional vectors may be concatenated over all sample points to obtain constant size feature vectors across users. For example, with k sample points, trajectory samples are obtained L/k meters apart. These samples are concatenated into a feature vector f of size k* 20 for further analysis.

Prior art [Chang, L. and Satava, R M and Pellegrini, C A and Sinanan, M N. Robotic surgery: identifying the learning curve through objective measurement of skill. Surgical endoscopy, 17(11):1744-1748, 2003; Kaul, S. and Shah, N. L. and Menon, M. Learning curve using robotic surgery. Current Urology Reports, 7(2):125-129, 2006; Lin, H. C. and Shafran, I. and Yuh, D. and Hager, G. D. Towards automatic skill evaluation: Detection and segmentation of robot-assisted surgical motions. Computer Aided Surgery, 11(5):220-230, 2006; Roberts, K. E. and Bell, R. L. and Duffy, A. J. Evolution of surgical skills training. World Journal of Gastroenterology, 12(20):3219, 2006] has always used motion data in the camera reference frame for further statistical analysis due to the absence of an alternative. The availability of corresponding trajectories, task constraints, and virtual models in the same space allows us to transform the experimental data to a reference frame in any other selected trial, at any given sample point. One axis of this reference frame is aligned along the local tangent of the trajectory, and the other two are placed in a fixed orthogonal plane. This creates a “trajectory space” that relates the task executions with respect to distances from the selected trial at a sample point, instead of with respect to a fixed endoscopic camera frame or static world frame over the entire trial.

A candidate trajectory e={e₁, e₂, . . . , eₖ} may be selected as the reference trajectory. Given any other trajectory u, for each pair of corresponding points, eₜ and u, calculate a homogeneous transformation T= [symbol] Rᵢ,pᵢ [symbol] may be calculated such that:

[symbol]

Rᵢ,pᵢ [symbol] eᵢ=uᵢ (3)

Similarly the velocity at a sample i, was obtained as:

vᵤᵢ=vᵤᵢ−vₑᵢ (4)

Finally the gripper angle gᵤᵢ was adjusted as gᵤᵢ−gₑᵢ. In trajectory space, the 10 dimensional feature vector for each instrument consists of {pᵢ, rᵢ, vᵤᵢ, gᵢ}. The candidate trajectory e may be an expert trial, or an optimal ground truth trajectory that may be available for certain simulated tasks, and can be computed for our experimental data. As an optimal trajectory lacks any relationship to a currently practiced proficient technique, we used an expert trial in the experiments reported here. Trials were annotated by the skill level of the subject for supervised statistical classification.

Multiple binary classifiers may be trained on experimental data. Fixed size uniformly sampled feature vectors permit a range of supervised classification approaches. Support vector machines (SVM) [Duda, Richard O. and Hart, Peter E. and Stork, David G. Pattern Classification (2nd Edition). Wiley-Interscience, 2000] may be used. SVMs are commonly used to classify observations into two classes (proficient vs. trainee).

SVM classification uses a kernel function to transform the input data, and an optimization step then estimates a separating surface with maximum separation. Trials represented by feature vectors (x) are divided into a training set and test set. Using the training set, an optimization method (Sequential Minimal Optimization) is employed to find support vectors sⱼ, weights αᵢ and bias b, which minimizes the classification error and maximizes the geometric margin. The classification is done by calculating c for an x is the feature vector of a trial belonging to the test set.

$$c = {\sum }_{j}^{ } {α}_{i} k ( {s}_{j} , x ) + b \qquad ( 5 )$$

where k is the kernel. Commonly employed Gaussian radial basis function (RBF) kernels may be used.

Given a trained classifier, its performance can be evaluated on held-out test data and common measures of performance can then be computed as:

$$\begin{matrix}precision = \frac{tp}{tp + fp} & ( 6 ) \\ recall = \frac{tp}{tp + fn} & ( 7 ) \\ accuracy = \frac{tp + tn}{tp + tn + fp + fn} & ( 8 )\end{matrix}$$

where tp are the true positives (proficient classified as proficient), to are the true negatives, fp are false positives, and fn are false negative classifications respectively.

Since the simulator is a new training environment, there is no validated definition of a proficient user yet. Several different methods of assigning the skill level for a trial were explored. To understand if there is any agreement between these different rating schemes, we calculated the Cohen's κ [Cohen, Jacob. A Coefficient of Agreement for Nominal Scales. Educational and Psychological Measurement, 20(1):37-46, 1960] which is a statistical measure of inter-rater agreement κ is calculated as follows:

$$κ = \frac{Pr ( a ) - Pr ( e )}{1 - Pr ( e )} \qquad ( 9 )$$

where Pr(a) is the relative observed agreement among raters and Pr(e) is the hypothetical probability of chance agreement. If the raters are in complete agreement κ is 1. If there is no agreement then κ≦0 The κ was calculated between the self-reported skill levels assumed to be the ground truth, and the classification produced by the methods above.

The C4.5 decision tree algorithm and SVM implementations in the Weka (Waikato Environment for Knowledge Analysis, University of Waikato, New Zealand) open source Java toolbox [Hall, M and Frank, E and Holmes, G and Pfahringer, B and Reutemann, P and Witten, I. H. The WEKA Data Mining Software: An Update. SIGKDD Explorations, 11, 2009] may be used for the following experiments. All processing was performed on a dual core workstation with 4 GB RAM.

### III. Experiments

These methods may be used to analyze dexterous tasks which simulate surgical exploration, and which require multiple system adjustments and significant pose changes for a successful completion, since these tasks which best differentiate between proficient and and trainee users. The simulation suite contains a wide range of dexterous training and surgical analog tasks.

A “pegboard ring maneuver” task which is a common pick and place task, and a “ring walk” task which simulates a vessel exploration in surgery from the simulation suite for the following experiments is selected.

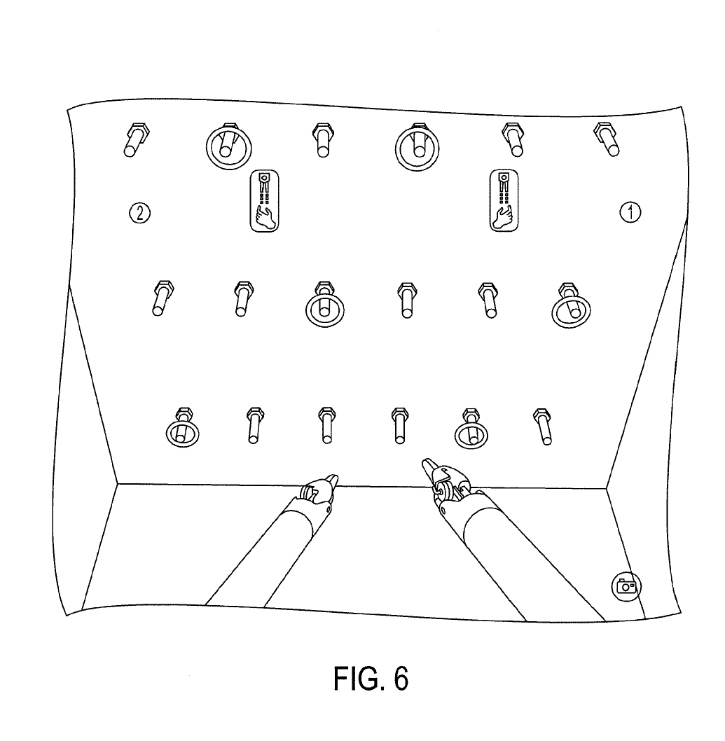

FIG. 6 illustrates a pegboard task according to an embodiment of the current invention. A pegboard task with the da Vinci Skills Simulator requires a set of rings to be moved to multiple targets. A user is required to move a set of rings sequentially from one set of vertical pegs on a simulated task board to horizontal pegs extending from a wall of the task board. The task is performed in a specific sequence with both the source and target pegs constrained (and presented as targets) at each task step. A second level of difficulty (Level 2) may be used.

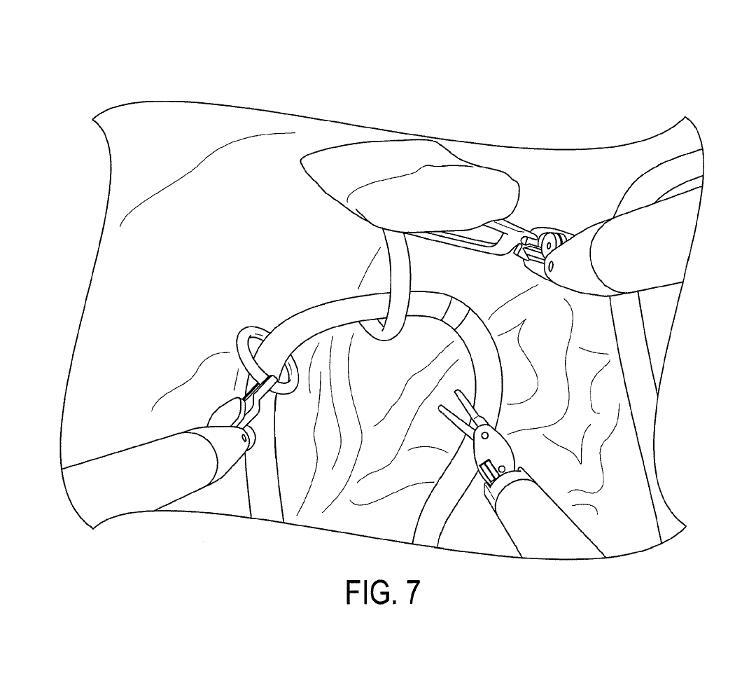

FIG. 7 illustrates a ring walk task according to an embodiment of the current invention. A ringwalk task with the da Vinci Skills Simulator requires a ring to be moved to multiple targets along a simulated vessel. A user is required to move a ring placed around a simulated vessel to presented targets along the simulated vessel while avoiding obstacles. The obstacles need to be manipulated to ensure successful completion. The task ends when the user navigates the ring to the last target. This task can be configured in several levels of difficulty, each with an increasingly complex path. A highest difficulty available (Level 3) may be used.

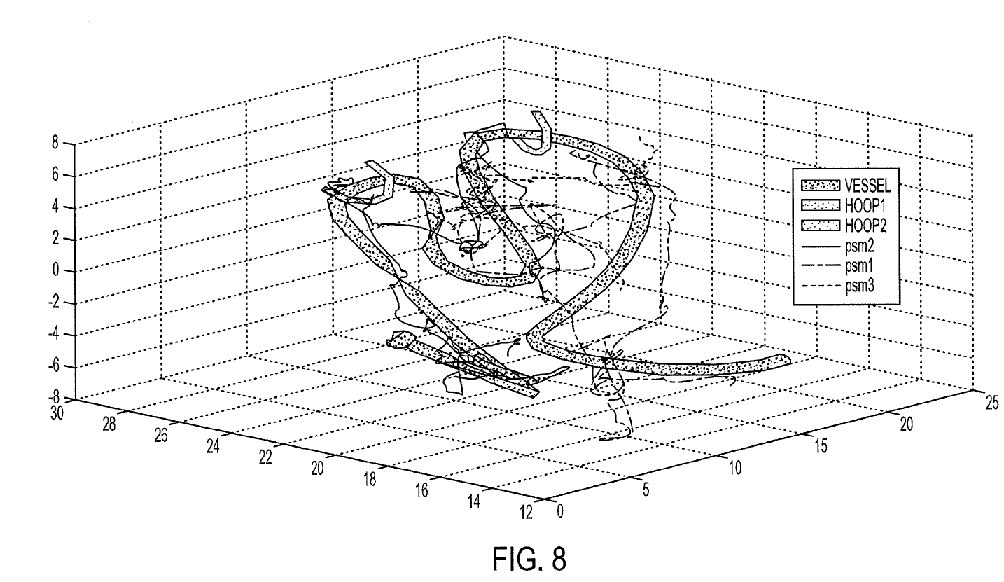

FIG. 8 illustrates task trajectories during the ring walk task according to an embodiment of the current invention. The gray structure is a simulated blood vessel. The other trajectories represent the motion of three instruments. The third instrument may be used only to move the obstacle. Thus, only the left and right instruments may be considered in the statistical analysis.

Experimental data was collected for multiple trials of these tasks from 17 subjects. Experimental subjects were the manufacturers' employees with varying exposure to robotic surgery systems and the simulation environment. Each subject was required to perform six training tasks in an order of increasing difficulty. The pegboard task was performed second in the sequence while the ringwalk task, the most difficult, was performed the last. Total time allowed for each sequence was fixed, so not all subjects were able to complete all six exercises.

Each subject was assigned a proficiency level on the basis of an initial skill assessment. Users with less than 40 hours of combined system exposure (9 of 17, simulation platform and robotic surgery system) were labeled as trainees. The remaining subjects, who had varied development and clinical experience and were considered proficient. Given that this is a new system still being validated, the skill level for a “proficient” user is arguable. In related work, alternative methodologies for classifying users as experts for the simulator and on real robotic surgery data were explored. For example, using structured assessment of a user's trials by an expert instead of self-reported data used here.

The emphasis of the results is not on the training of the classifier but rather on using alternative transformation spaces and then classifying skill. Therefore, the establishment of the ground truth may not be a weakness of the methods proposed. Any method for assignment of skill level, and in training of our classifiers, may be used. Reports in prior art, e.g. [Judkins, T. N. and Oleynikov, D. and Stergiou, N. Objective evaluation of expert and novice performance during robotic surgical training tasks. Surgical Endoscopy, 23(3):590-597, 2009], show that a relatively short training period is required for competency in ab initio training tasks. This, however, may also be due to the lack of discriminating power in the metrics used, or lack of complexity in the experimental tasks.

TABLE 1
The Experimental dataset consisted
of multiple trials from two tasks.

| | Proficient | Trainee | |
|---|---|---|---|
| Task | Trials | Trials | Total |
| Ringwalk | 22 | 19 | 41 |
| Pegboard | 24 | 27 | 51 |

First the metrics in the scoring system integrated in the da Vinci Skills Simulator are investigated. The list of metrics includes:
Economy of motion (total distance traveled by the instruments)
Total time
Excessive force used
Instrument collisions
Total out of view instrument motion
Range of the master motion (diameter of the master manipulator bounding sphere)
Critical errors (ring drop etc.)

There was no adaptive threshold which could separate the experts from the novices with an acceptable accuracy (>85% across tasks) based on the above individual metrics. Given values s₁, s₂, . . . , s_{M} units for M metrics m₁, m₂, . . . , m_{M}. The simulator first computes a scaled score fⱼ for each metric:

$${f}_{{m}_{j}} = \frac{( {s}_{j} - {l}_{j} ) \times 100}{{u}_{j} - {l}_{j}} \qquad ( 10 )$$

where the upper and lower bounds are based on the developers' best guesses to be uⱼ and lⱼ, and a final weighted score f:

$$f = {\sum }_{i = 1}^{M} {w}_{i} {f}_{i} \qquad ( 11 )$$

In the current scoring system, all the weights are equal and Σᵢ₌₁^{M}wᵢ=1. One aim was to improve the scoring system in a way which would differentiate between experts and novices better.

Unequal weights may be assigned to the individual metrics, based on their relative importance computed as separation of trainee and expert averages. Let for a particular metric mⱼ, μ_{E}_{ⱼ} and μ_{N}_{ⱼ} be the expert and the novice mean values calculated from the data. Let σ_{E}_{ⱼ} be the expert standard deviation. The new weight ŵⱼ may be assigned to be:

$${\hat{w}}_{j} = \frac{{μ}_{{E}_{j}} - {μ}_{{N}_{j}}}{{σ}_{{E}_{j}}} \qquad ( 12 )$$

ŵⱼ were normalized so that Σᵢ₌₁^{M}ŵᵢ=1. The upper bound on performance was modified to ûⱼ

ûⱼ=μ_{E}_{ⱼ}+3σ_{E}_{ⱼ} (13)

if experts were expected to have higher values for that metric, and otherwise to

ûⱼ=μ_{E}_{ⱼ}−3σ_{E}_{ⱼ} (14)

Similarly, the lower bound was modified to

\hat{l}ⱼ=μ_{N}_{ⱼ}−σ_{N}_{ⱼ} (15)

if experts are expected to have higher values for that metric, and otherwise to

ûⱼ=μ_{N}_{ⱼ}+σ_{N}_{ⱼ} (16)

The performance of this weighted scoring system with the current system may be compared by comparing how well they differentiated between proficient and trainee users. Performance of classification based on the current scheme is shown in Table 2 along with that of the new scoring system. While the improved scoring system performed acceptably for simple tasks (pegboad), accuracy (77%) was still not adequate for complex tasks such as the ringwalk

TABLE 2
Classification accuracy and corresponding
thresholds for task scores.

| Task | Th_{curr} (%) | Acc_{curr} (%) | Th_{new} | ACC_{new} |
|---|---|---|---|---|
| Ringwalk | 56.77 | 73.17 | 75.54 | 77.27 |
| Pegboard | 95.44 | 78.43 | 65.20 | 87.03 |

Adaptive threshold computations were also useful on some basic metrics. These included economy of motion, and total time, as the proficient and trainee means were well separated. However, Tables 3 and 4 show that distance and time are poor metrics for distinguishing skill levels.

TABLE 3
Classification accuracy and corresponding
thresholds instrument tip distance.

| Task | p_{D} Threshold (cm) | Accuracy (%) |
|---|---|---|
| Ringwalk | 40.26 | 52.5 |
| Pegboard | 23.14 | 72 |

TABLE 4
Classification accuracy and corresponding thresholds for
the time required to successfully complete the task.

| Task | p_{T} Threshold (seconds) | Accuracy (%) |
|---|---|---|
| Ringwalk | 969 | 52.5 |
| Pegboard | 595 | 68 |

The ribbon measure R_{A} is also calculated. An adaptive threshold on this pose metric outperforms adaptive thresholds on the simple metrics above for skill classification. Tables 5, 6 report this baseline performance.

TABLE 5
Classification accuracy and corresponding thresholds
for the R_{A} measure for the ringwalk task.

| Manipulator | R_{A} Threshold (cm²) | Accuracy (%) |
|---|---|---|
| Left | 128.8 | 80 |
| Right | 132.8 | 77.5 |

TABLE 6
Classification accuracy and corresponding thresholds for the
R_{A} measure for left and right instruments for the pegboard task.

| Manipulator | R_{A} Threshold (cm²) | Accuracy (%) |
|---|---|---|
| Left | 132.9 | 80 |
| Right | 107.6 | 78 |

Cohen's kappa [Cohen, Jacob. A Coefficient of Agreement for Nominal Scales. Educational and Psychological Measurement, 20(1):37-46, 1960] was also calculated for the skill classification to identify agreement with the ground truth labels. The results show that the ribbon metric reaches the highest agreement with the ground truth labeling (Table 6), where as the distance and time don't have a high agreement among themselves. The numbers p1D-time and p2D-time for ringwalk are undefined because the classification is the same label for both criteria.

TABLE 7
Cohen's κ for classification based on different metrics
vs. ground truth (GT). P1/2 is the left/right instrument, D
the distance traveled, T the task time, and R the ribbon metric.

| Task | Rater pairs | κ |
|---|---|---|
| 5*Pegboard | p1D-GT | 0.40 |
| | p2-D-GT | 0.41 |
| | time-GT | 0.34 |
| | p1R-GT | 0.60 |
| | p2R-GT | 0.55 |
| | p1D-time | 0.21 |
| | p2D-time | 0.10 |
| 5*Ringwalk | p1D-GT | 0.0 |
| | p2D-GT | 0.0 |
| | time-GT | 0.0 |
| | p1R-GT | 0.59 |
| | p2R-GT | 0.53 |
| | p1D-time | undefined |
| | p2D-time | undefined |

TABLE 8
Binary classification performance of motion classification in
the “trajectory” space for the Ring Walk task

| Task | k | Precision (%) | Recall (%) | Accuracy (%) |
|---|---|---|---|---|
| 3*Pegboard | 32 | 81 | 65.4 | 74 |
| | 64 | 92.0 | 88.5 | 90.0 |
| | 128 | 83.9 | 100.0 | 90.0 |
| 3*Ringwalk | 32 | 88.9 | 84.2 | 87.5 |
| | 64 | 86.7 | 68.4 | 80.0 |
| | 128 | 87.5 | 73.7 | 82.5 |

Statistical classification: Each API motion trajectory (in the fixed world frame) was sampled at k={32,64,128} points which provided feature vectors of fᵢ of 640,1280,2560 dimensions. 41 trials for the ringwalk and, and 51 trials of the pegboard task from the 17 subjects were conducted.

Binary SVM classifiers were trained using Gaussian radial basis function kernels and performed a k-fold cross-validation with the trained classifier to calculate the precision, recall, and accuracy. Table 9 shows the classification results in the static world frame do not outperform the baseline ribbon metric computations.

TABLE 9
Performance of binary SVM classification (expert
vs. novice) in the world frame for both tasks.

| Task | k | Precision (%) | Recall (%) | Accuracy (%) |
|---|---|---|---|---|
| 3*Pegboard | 32 | 69.0 | 76.9 | 70.0 |
| | 64 | 75.8 | 96.2 | 82.0 |
| | 128 | 73.5 | 96.2 | 80.0 |
| 3*Ringwalk | 32 | 66.7 | 63.2 | 67.5 |
| | 64 | 63.2 | 63.2 | 65 |
| | 128 | 64.7 | 57.9 | 65 |

Binary SVM classifiers using the “trajectory” space feature vectors outperformed all other metrics. Table 8 includes these classification results. The trajectory space distinguishes proficient and trainee users with a 87.5% accuracy (and a high 84.2% recall) with 32 samples, which is comparable to the art [Rosen, J. and Hannaford, B. and Richards, C. G. and Sinanan, M. N. Markov modeling of minimally invasive surgery based on tool/tissue interaction and force/torque signatures for evaluating surgical skills. IEEE Transactions on Biomedical Engineering, 48(5):579-591, 2001] for real robotic surgical system motion data. Larger number of samples reduce this performance due to extra variability. Similar small performance changes are seen with alternate choice of candidate trajectories.

### IV. Conclusions and Future Work

Simulation based robotic surgery training is being rapidly adopted with the availability of several training platforms. New metrics and methods for proficiency classification (proficient vs. trainee) are reported based on motion data from robotic surgery training in a simulation environment. Such tests are needed to report when a subject may have acquired sufficient skills, and would pave the way for a more efficient, and customizable proficiency based training instead of current fixed time or trial count training paradigms.

Compared to a classification accuracy of 67.5% using raw instrument motion data, a decision tree based thresholding of a pose “ribbon area” metric provides 80% baseline accuracy. Working in the trajectory space of an expert further improves these results to 87.5%. These results are comparable to the accuracy of skill classification reported in the art (e.g [Rosen, J. and Hannaford, B. and Richards, C. G. and Sinanan, M. N. Markov modeling of minimally invasive surgery based on tool/tissue interaction and force/torque signatures for evaluating surgical skills. IEEE Transactions on Biomedical Engineering, 48(5):579-591, 2001]) with other motion data.

In contrast to real environments, the ground truth for the environment is accurately known in the simulator. The work may be extended to use the ground truth location of the simulated vessel together with the expert trajectory space results reported here. The work described also used a portion of experimental data obtained from the manufacturers employees.

A binary classifier on entire task trajectories is used here, while noting that distinctions between users of varying skills are highlighted in task portions of high curvature/dexterity. Alternative classification methods and different trajectory segmentation emphasizing portions requiring high skill may also be used. Data may also be intelligently segmented to further improve classification accuracy.

Lastly, in related work on real da Vinci surgical system motion data, man-machine interaction may be assessted [Kumar, R and Jog, A and Malpani, A and Vagvolgyi, B and Yuh, D and Nguyen, H and Hager, G and Chen, C C G. System operation skills in robotic surgery trainees. The International Journal of Medical Robotics and Computer Assisted Surgery, :accepted, 2011; Yuh, D D and Jog, A and Kumar, R. Automated Skill Assessment for Robotic Surgical Training 47th Annual Meeting of the Society of Thoracic Surgeons, San Diego, Calif., pages poster, 2011] via another related study. Additional similar methods of data segmentation, analysis, and classification for simulated data are also currently in development.

We claim:

1. A computer-implemented method of analyzing a sample task trajectory comprising:
obtaining, with one or more computers, position information of an instrument in the sample task trajectory;
obtaining, with the one or more computers, pose information of the instrument in the sample task trajectory;
comparing, with the one or more computers, the position information and the pose information for the sample task trajectory with reference position information and reference pose information of the instrument for a reference task trajectory;
determining, with the one or more computers, a skill assessment for the sample task trajectory based on the comparison; and
outputting, with the one or more computers, the determined skill assessment for the sample task trajectory.

2. The computer-implemented method of claim 1, wherein the sample task trajectory comprises a trajectory of the instrument during a surgical task, wherein the instrument comprises a simulated surgical instrument of a surgical robot.

3. The computer-implemented method of claim 1, wherein pose information represents roll, pitch, and yaw information of the instrument.

4. The computer-implemented method of claim 3, wherein the pose information of the instrument is represented using at least one of:
a position vector and a rotation matrix in a conventional homogeneous transformation framework;
three angles of pose and three elements of a position vector in a standard axis-angle representation; or
a screw axis representation.

5. The computer-implemented method of claim 1, wherein comparing the position information comprises:
transforming the position information of the instrument and the pose information of the instrument from a coordinate system based on camera views in the sample task trajectory of a camera of a robot including the instrument to at least one of:
a coordinate system based on the reference task trajectory; or
a coordinate system based on a world space.

6. The computer-implemented method of claim 1, wherein comparing comprises:
calculating surface area spanned by a line along an instrument axis of the instrument during the sample task trajectory; and
comparing the calculated surface area with a corresponding surface area spanned during the reference task trajectory.

7. The computer-implemented method of claim 6, wherein calculating the surface area comprises generating a sum of areas of consecutive quadrilaterals defined by the line sampled at one or more of:
time intervals;
equal instrument tip distances; or
equal angular or pose separation.

8. The computer-implemented method of claim 1, wherein obtaining the position information and the pose information comprises filtering the position information and the pose information based on detecting the importance or task relevance of the position information and the pose information.

9. The computer-implemented method of claim 8, wherein detecting the importance or task relevance is based on at least one of:
detecting a portion of the sample task trajectory which is outside a field of view; or
identifying a portion of the sample task trajectory which is unrelated to a task.

10. The computer-implemented method of claim 1, wherein determining a skill assessment comprises classifying the sample task trajectory into a binary skill classification for users of a surgical robot based on the comparison.

11. The computer-implemented method of claim 1, further comprising:
obtaining velocity information of the instrument in the sample task trajectory; and
obtaining gripper angle information of the instrument in the sample trajectory,
wherein comparing the position information and the pose information further comprises comparing the velocity information and gripper angle information with reference velocity information and reference gripper angle information of the instrument for the reference task trajectory.

12. A system for analyzing a sample task trajectory comprising:
a controller configured to receive motion input from a user for an instrument for the sample task trajectory;
a display configured to output a view based on the received motion input;
a processor configured to:
obtain position information of the instrument in the sample task trajectory based on the received motion input;
obtain pose information of the instrument in the sample task trajectory based on the received motion input;
compare the position information and the pose information for the sample task trajectory with reference position information and reference pose information of the instrument for a reference task trajectory;
determine a skill assessment for the sample task trajectory based on the comparison; and
output the skill assessment.

13. The system for analyzing, further comprising:
a simulator configured to simulate the sample task trajectory during a surgical task based on the received motion input and simulate the view based on the sample task trajectory.

14. The computer-implemented method of claim 1, wherein pose information represents roll, pitch, and yaw information of the instrument.

15. The computer-implemented method of claim 12, wherein comparing the position information comprises:
transforming the position information of the instrument and the pose information of the instrument from a coordinate system based on camera views in the sample task trajectory of a camera of a robot including the instrument to at least one of:
a coordinate system based on the reference task trajectory; or
a coordinate system based on a world space.

16. The computer-implemented method of claim 12, wherein comparing comprises:
calculating surface area spanned by a line along an instrument axis of the instrument during the sample task trajectory; and
comparing the calculated portion surface area with a corresponding surface area spanned during the reference task trajectory.

17. The computer-implemented method of claim 12, wherein obtaining the position information and the pose information comprises filtering the position information and the pose information based on detecting the importance or task relevance of the position information and the pose information.

18. The computer-implemented method of claim 12, wherein determining a skill assessment comprises classifying the sample task trajectory into a binary skill classification for users of a surgical robot based on the comparison.

19. The computer-implemented method of claim 12, further comprising:
obtaining velocity information of the instrument in the sample task trajectory; and
obtaining gripper angle information of the instrument in the sample trajectory,
wherein comparing the position information and the pose information further comprises comparing the velocity information and gripper angle information with reference velocity information and reference gripper angle information of the instrument for the reference task trajectory.

20. One or more tangible non-transitory computer-readable storage media for storing computer-executable instructions executable by processing logic, the media storing one or more instructions for:
obtaining position information of an instrument in the sample task trajectory;
obtaining pose information of the instrument in the sample task trajectory;
comparing the position information and the pose information for the sample task trajectory with reference position information and reference pose information of the instrument for a reference task trajectory;
determining a skill assessment for the sample task trajectory based on the comparison; and
outputting the skill assessment for the sample task trajectory.
